(19) United States
(12) Patent Application Publication
(10) Pub. No.: US 2007/0055129 A1
(43) Pub. Date: Mar. 8, 2007

(54) Method and device for displaying a surgical instrument during placement thereof in a patient during a treatment

(75) Inventor: Rainer Graumann, Hochstadt (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengesellschaft

(21) Appl. No.: 11/510,236

(22) Filed: Aug. 24, 2006

(30) Foreign Application Priority Data
Aug. 24, 2005 (DE) 10 2005 040 049.3

(51) Int. Cl. A61B 5/05

(52) U.S. Cl. 600/407

(57) ABSTRACT

In a method and device for representation of a surgical instrument during placement thereof in a patient during a treatment, first 3D image data of the patient are generated before the treatment and in these a relevant body structure is identified. During the treatment, second 3D image data of the patient are generated. The body structure and the second 3D image data are associated with one another with spatial accuracy. During the placement of the instrument at least two x-ray images are generated from respectively different viewing directions and the instrument and the body structure are associated with one another with spatial accuracy. At least the instrument and the body structure are shown in a common image.

### BACKGROUND OF THE INVENTION

1. Field of the Invention

The present invention concerns a method and device for representation of a surgical instrument during placement thereof in a patient in a treatment procedure.

2. Description of the Prior Art

Minimally-invasive and non-invasive methods steadily increases both in medical diagnosis and therapy or treatment of patients (normally living people or animals). Surgical instruments are inserted (introduced) into the body of the patient. Such surgical instruments can be, for example, biopsy needles, RF ablation needles, drainage: tubes, drills, screws etc. Because the body of the patient is not subjected to open surgery, the doctor or operator of the instrument cannot view the medical or surgical instrument or its important part (for example its treatment tip). This problem can also sometime occur in open surgery, when the sight of the instrument, the treatment area or other body regions of interest of the patient is obscured for other reasons. Such viewing of the instrument, however, is desirable for the diagnosis or therapy.

Since in all of these cases a direct view of the instrument is denied, medical imaging is necessary. Only in this manner can an unerring and gentle placement of the instrument thus occur. As used herein “placement” means both the placement in the target area (thus for example the precise puncturing of a tumor) and the general direction of the instrument. The direction of the instrument to the target area should be executed in a manner that is optimally protective to the patient, meaning that risk structures (such as other organs, sensitive tissue etc.) should be avoided or be damaged as little as possible.

By means of the imaging, which ideally is implemented as a type of steady imaging corresponding to a television camera (also called online imaging), the treating doctor can track the placement of the instrument in real time, for example on a monitor.

Today, primarily computed tomography, 2D fluoroscopy and ultrasound are used for such imaging.

Classical 2D fluoroscopy has the disadvantage of supplying little or no soft tissue contrast at all. Only bone structures are detectable in such fluoroscopic images, and hardly any or no risk structures in the form of organs or tissue that could be harmed by the surgical instrument.

In ultrasound imaging, the problem alternatively or additionally occurs that both the general detectability of body structures (for example, target structure and risk structure) in the patient and their graphical representation are extremely difficult and user-dependent. Both the acquisition and the interpretation of ultrasound images for the aforementioned purpose require a large degree of experience and ability of the user. The advantage of the ultrasound imaging is the very good patient accessibility and the simple and cost-effective manageability of the necessary equipment and techniques.

In order to avoid the disadvantages of 2D fluoroscopy and ultrasound, it is also known to implement computed tomographic imaging during the placement of the surgical instrument (thus, for example, intraoperatively). This is presently effected by a stationary computed tomography apparatus. A significant disadvantage is that the patient must be inserted into the narrow tube of the computed tomography scanner for imaging. The patient accessibility is thus severely limited. Often no space or too little space is available for the instrument insertion, such that the patient must be frequently moved into and out of the scanner. This is in turn very time-consuming and cost-intensive due to the high acquisition and operating costs of a computed tomography apparatus. The flexibility in the usage of computed tomography is severely limited due to the aforementioned patient access etc. The advantage of using computed tomography is the good image quality, in particular in the soft tissue contrast and the short measurement times for acquisition of the image information (for example for CT fluoroscopy).

### SUMMARY OF THE INVENTION

An object of the present invention is to improve methods and devices for representation of a surgical instrument during placement thereof in a patient in a treatment procedure.

With regard to the method, this object is achieved by a method for representation of a surgical instrument during placement thereof in a patient during a treatment, with the following steps:
a) first 3D image data of the patient that show at least one body structure of the patient that is relevant for the placement are generated before the treatment,
b) the body structure is identified in the first 3D image data,
c) second 3D image data of the patient that enable at least one spatially-accurate association of the second 3D image data with the first 3D image data are generated with an x-ray system during the treatment,
d) the body structure and the second 3D image data are associated with one another with spatial accuracy,
e) during the placement of the instrument at least two x-ray images of the patient with the instrument are generated with the x-ray system from respective varying viewing directions in known position relative to the second 3D image data,
f) the instrument and the body structure are associated with one another with spatial accuracy using the known relative position and the second 3D image data, and at least the instrument and the body structure are shown in a common image.

First 3D image data of the patient are thus initially generated in step a). The first point in time is selected such that it is still not decisive how good the patient access is. An imaging method is therefore selected for generating the 3D image data that achieves the best possible results in the imaging in order to show desired body structures (namely those relevant for the placement) of the patient. As mentioned above, such body structures are, for example, organs, bones, tissue or the like in which the instrument is to be placed or which are otherwise relevant during the treatment. For example, in the usage of biopsy needles damage to a healthy organ of the patient must be unconditionally prevented. In this case this organ and the target area in the patient would be body structures relevant for the placement of the biopsy needle as a surgical instrument.

In this context, “treatment” can also be a diagnosis or the like.

The body structure is therefore identified in the high-quality first 3D image data, meaning that, for example, its size or position or shape are determined and, for example, emphasized for a viewer by color or in high contrast in the first 3D image data. Segmentation of the appertaining body structure for this purpose is suitable.

According to the invention an x-ray system that generates second 3D image data of the patient is now used during the treatment. An inexpensive x-ray system offering a good patient access is used which, under the circumstances, is not suited or not well-suited to identify the appertaining body structures. The second x-ray system, however, enables at least a spatially-accurate association of the second 3D image data with the first 3D image data.

The position of the second 3D image data relative to the x-ray system is naturally known, for example due to the corresponding calibration, adjustment of corresponding projection matrices, etc. which are innate to the x-ray system. The spatial coordinates of the second 3D image data (also, for example, with regard to the patient) are thus known in the coordinate system of the x-ray system (which typically rests in the treatment space in which the treatment of the patient is implemented). The spatial coordinates of the first 3D image data are also currently known due to the spatially-accurate association, thus a valid coordinate system is known during the treatment and with this the spatial coordinates of the relevant body structure of the patient.

This spatial association is thus inventively achieved without the usage of external or internal patient markers or the like for spatial association of the body structure at the moment of the treatment, thus for its localization. A 3D image data set hereby allows a significantly more precise spatial association than the use of individual 2D x-ray exposures or a set of 2D x-ray exposures.

Moreover, further x-ray images of the patient together with the instrument are nevertheless generated with the same x-ray system or a different x-ray system during the placement from respective varying viewing directions at known position relative to the second 3D image data. Due to the known relative position of the (normally two-dimensional) x-ray images of the patient with instrument here a spatially-accurate association can also ensue between the instrument, the patient, the second 3D image data and therewith the first 3D image data and body structure. As a result, it is possible to show the instrument together with the body structure in a common image. Individual x-ray images are hereby sufficient for placement since now essentially only the instrument is still moving in the patient. The body structures are viewed as stationary for the duration of the treatment.

The individual or doctor implementing the treatment thus has available to him or her at least image information of the instrument guided by him or her and the relevant body structures, both in real time and in the best possible quality, all of the image information being associated with one another with spatial accuracy. The image information then is displayed in a common image according to the invention. The doctor can then unerringly guide the treatment or placement of the instrument according to this display. Due to the x-ray system, the best-possible patient access, flexibility, easy manageability, cost-effectiveness etc. are additionally combined. Moreover, the entire medical system is simpler and more cost-effective because no external navigation system, and therefore no patient markers, is necessary.

The spatially-accurate association of the image information items with one another, also called registration, ensues by the fusing or spatial association of first and second 3D image data. This can be achieved with simple and cost-effective standard image processing hardware or software.

CT or MR image data can be generated in a stationary imaging system as the first 3D image data in step a). As mentioned above, expensive stationary angio-systems supply extremely high-quality first 3D image data so that the body structure is available in very high image quality for the method. Due to the utilization of the relevant part of the image data, namely the body structures, this quality is also used for the placement of the instrument in the common image with the body structures.

X-ray image data as the second 3D image data can be generated with a 3D x-ray C-arm as an x-ray system in step c). Such 3D x-ray C-arms are generally available today anyway in treatment rooms, are relatively cost-effective, can be used flexibly, offer the best-possible patient access etc. If such a 3D x-ray C-arm is equipped with a planar image detector, this can even offer soft tissue resolution to a limited degree, which further simplifies and improves the spatial association of first and second 3D image data. Certain variations in the body structures, for example due to changed body position of the patient given the acquisition of first and second 3D image data, thus can be corrected to a certain extent, which further increases the representation of the body structure in the common image and therewith the reliability in the placement of the surgical instrument.

In step e) the x-ray images can be acquired with the same 3D x-ray C-arm as in step c) without displacing (relocating) the C-arm between the steps c) and e). Since the same x-ray C-arm is used, the number of the devices to be used in the treatment decreases and with it the costs and the effort. Since the x-ray C-arm is not displaced, the same projection matrices or coordinate associations between imaging and patient apply for this x-ray C-arm relative to the patient in both of the method steps cited above. The spatially-accurate association of the x-ray images generated by instrument and patient in step e) with the second 3D image data etc. is then particularly simple, or is inherent. This also incidentally applies when the 3D x-ray C-arm is positioned in the exact same position at least in the steps c) and e). This can, for example, occur via a locking in the floor of the treatment room in a predetermined acquisition. In spite of this the x-ray C-arm can be moved from the patient or from the treatment area in between in order to further increase the patient access. Upon moving back into the previous position for further imaging the coordinate association is again automatically ensured.

Alternatively, a simple and cost-effective navigation or tracking system can be installed on the 3D x-ray C-arm that tracks its position, for example in the treatment space relative to the patient. The known relative position between the x-ray images and the second 3D image data in step e) is thereby likewise achieved.

In step f) the body structure can be mixed (superimposed) into at least one of the x-ray images generated in step e) as a common image. X-ray images are thus available to the doctor in the accustomed manner, in which x-ray images he or she detects the current position of the surgical instrument, but in accordance with the invention this is improved by the high-quality image content of the relevant body structure of the patient. The doctor thereby obtains additional image information that he or she can additionally evaluate and use, for example in a critical treatment phase.

With regard to the device the above object is achieved in accordance with the invention by a device for representation of a surgical instrument during placement thereof in a patient during a treatment, with an interface for importation of first 3D image data of the patient generated before the treatment, the first 3D image data show at least one body structure of the patient that is relevant for the placement; and with an x-ray system for generation of second 3D image data of the patient during the treatment, the second 3D image data enabling at least one spatially-accurate association of the second 3D image data with the first 3D image data, and for generation of at least two x-ray images of the patient with the instrument from respectively different viewing directions in known positions relative to the second 3D image data during the placement of the instrument. An evaluation unit identifies the body structure in the first 3D image data and spatially-accurately associates the body structure and the second 3D image data relative to one another and spatially-accurately associates the instrument and the body structure using the known relative position and the second 3D image data relative to one another. A display unit displays at least the instrument and the body structure in a common image.

The inventive device has the same advantages that have already been explained in detail in connection with the inventive method. The interface for importation of the first 2D image data can be, for example, a network interface to a hospital information system, a CD-ROM reader or the like. The evaluation unit can be a separate device, for example in the form of a workstation or special hardware which is suitable for execution of the inventive method, or be implemented as auxiliary hardware or software in a sub-device of the inventive device, for example the x-ray system, or its system controller. The display unit likewise can be a separate screen, monitor or a monitor of the x-ray system, the hospital information system or the like that is used for this and that is already located the treatment room.

The x-ray system can be a 3D x-ray C-arm system. The advantages thereof have already been explained above.

### DESCRIPTIONI OF THE DRAWINGS

FIG. 1 schematically illustrates a patient in a magnetic resonance tomography before beginning a biopsy in accordance with the present invention.

FIG. 2 schematically illustrates the patient of FIG. 1 in a treatment room with x-ray system together with a doctor during a biopsy in accordance with the present invention.

### DESCRIPTION OF THE PREFERRED EMBODIMENTS

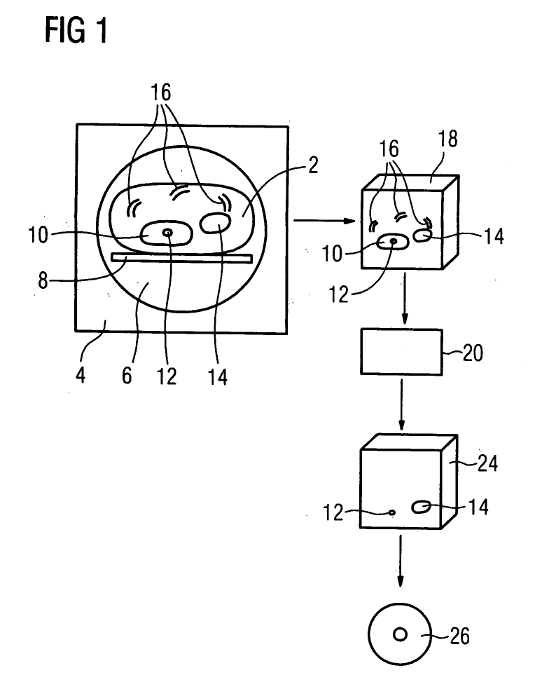

FIG. 1 shows in cross-section a patient 2 in a magnetic resonance tomograph 4. The patient lies in the dorsal position on a patient bed 8 inserted into the tube 6 of the magnetic resonance tomograph 4. Schematically indicated inside the patient 2 are the liver 10 with a tumor 12 located therein, a kidney 14 and a bone structure or bone 16. FIG. 1 shows the MR examination of the patient 2 at a first point in time at which the patient has sought a doctor (not shown) due to painful discomfort in the abdomen, and the doctor has immediately implemented a diagnosis of the patient 2 using the magnetic resonance tomography apparatus 4. For this purpose, the doctor generates an MR exposure in the form of an MR reconstruction volume 18 with the magnetic resonance tomography apparatus 4. Due to the high-quality imaging in the magnetic resonance tomography apparatus 4, the MR reconstruction volume 18 embodies a complete mapping of the inside of the patient 2, namely the liver 10, a tumor 12, the kidney 14 and the bone 16. According to the invention, the diagnosis occurs at the first point in time.

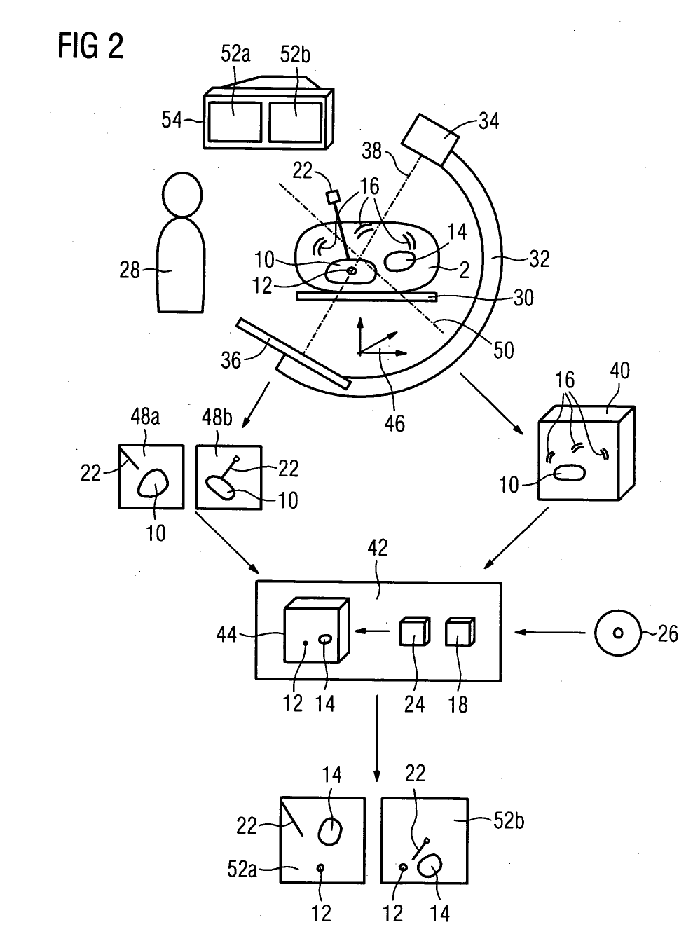

Since the doctor has diagnosed the tumor 12, a biopsy is scheduled to confirm this suspicion, which biopsy is to be implemented at a later point in time, for example two weeks after the diagnosis shown in FIG. 1. In preparation for the biopsy to be implemented, in the MR reconstruction volume 18 the doctor segments the subjects necessary or relevant for the biopsy (namely the tumor 12 and the kidney 14) at a computer workstation 20. The kidney 14 represents a risk structure for the biopsy to be implemented since it must not be damaged in the biopsy by the medical instrument (namely the biopsy needle 22 shown in FIG. 2). These body structures are included in a segmentation result 24 according to the invention.

The doctor stores the segmentation result 24 including the tumor 12 and the kidney 14 as a graphical representation together with the MR reconstruction volume 18 on a CD-ROM 26. These data correspond to the inventive first 3D image data.

At a later point in time, thus two weeks after implementation of the diagnosis according to FIG. 1, the patient 2 undergoes the biopsy at a different doctor 28. The biopsy according to FIG. 2 is the treatment according to the invention and is implemented at a different location than the diagnosis according to FIG. 1. In the biopsy the patient 2 also reclines on a patient bed 30 that is a part of an x-ray C-arm system 32 having an isocenter in which the patient 2 rests. The x-ray C-arm 32 has an x-ray source 34 and a planar image detector 36 or alternatively an x-ray image intensifier (not shown) in a known manner. The x-ray radiation emitted from the x-ray source 34 toward the planar image detector 36 is represented by a central ray 38 and corresponds to the viewing direction of an x-ray image to be acquired.

At the beginning of the biopsy the doctor 28 now produces numerous x-ray images (not shown) of the patient 2 by a 3D C-arm imaging, the x-ray images being generated by isocentric movement of the x-ray C-arm 32 around the patient 2 in a known manner. A 3D C-arm reconstruction volume 40 is calculated in a known manner from the multiple x-ray images. This 3D C-arm reconstruction volume 40 corresponds to the inventive second 3D image data. Due to the notably poorer soft tissue contrast of the x-ray C-arm 32 relative to the magnetic resonance tomography apparatus 4, the 3D C-arm reconstruction volume 40 merely shows mappings of the bones 16 and the liver 10 of the patient 2. The tumor 12 as well as the kidney 14 is not able to be detected in the 3D C-arm reconstruction volume 40.

The spatial association of the aforementioned MR reconstruction volume 18 relative to the current position of the patient or relative to a fixed coordinate system 46 in treatment space is still unknown.

For this reason, the doctor 28 loads the data stored on the CD-ROM 26 (namely the MR reconstruction volume 18 and the segmentation result 24) into a computer workstation 42. In the computer workstation 42, by image processing algorithms the MR reconstruction volume 18 is associated with spatial accuracy with the 3D C-arm reconstruction volume 40 using the bone 16 and the liver 10, thus with the image content of the respective image data. The segmentation result 24 is likewise associated with spatial accuracy since it was determined from the MR reconstruction volume 18 in known relative position (which MR reconstruction volume 18 is likewise stored on the CD-ROM 26), such that a total image data set 44 comprising all previous information is created. This includes the segmentation result 24, thus the graphically-emphasized tumor 12 and the kidney 14, so the total image data set 44 is now known with accurate coordinates in the coordinate system 46 (in which the patient 2 now rests) defined by the x-ray C-arm 32.

The doctor 28 now begins to insert the biopsy needle 22 into the patient 2. Two x-ray images 48a and 48b of the patient 2 with the biopsy needle 22 are respectively continuously produced in two different viewing directions, indicated by the central ray 38 and the central ray 50 displaced relative to this. Only one pair of these exposures is exemplarily shown in FIG. 2. Due to the lacking soft tissue contrast, the biopsy needle 22 and the liver 10 of the patient are visible in each of the images 48a and 48b, but not the tumor 12 or the kidney 14.

The x-ray exposures 48a and 48b are therefore acquired with the x-ray C-arm 32 that, although it is moved, is unchanged with regard to its location. The acquisition geometry or the projection matrices therefor are thus valid and unchanged with regard to that of the 3D C-arm reconstruction volume 40 (and thereby with regard to the total image data set 44), and the position of said projection matrices is therewith known relative to the x-ray exposures 48a and 48b.

The two x-ray images 48a and 48b are therefore linked with the total image data set 44, and in fact such that two artificial x-ray images 52a and 52b are generated in the viewing direction of the x-ray images 48a and 48b. The artificial x-ray images 52a and 52b show the biopsy needle 22 from the x-ray images 48a and 48b and the tumor 12 and the kidney 14 of the patient from the 3D reconstruction volume 18 or segmentation result 24. It is therefore possible for the doctor to place the biopsy needle 22 in the tumor 12 by observation of the artificial x-ray images 52a and 52b on a monitor 54 without damaging the kidney 14.

The liver 10 and the bone 16, the images of which the doctor 28 does not need for the placement of the biopsy needle 22 into the tumor 12, are masked in the x-ray images 52a.

An alternative is to also mix the biopsy needle 22 and tumor 12 and kidney 14 into the x-ray images 48a and 48b and to show these to the doctor 28 as long as the structures or image contents mapped in the x-ray images 48a and 48b do not interfere.

Another alternative is to acquire the image data set 40 and the x-ray images 48a and 48b with separate x-ray systems (not shown) rather than with the same C-arm 32. It is only necessary that their respective acquisition geometries (thus the spatial association between the imaged subject and the image content of the acquired images) be known as in the form of relations between the various apparatuses.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for displaying a surgical instrument during placement thereof in a patient during a treatment, comprising the steps of:
(a) before a medical treatment of a patient, obtaining first 3D image data of the patient showing a body structure of the patient relevant for subsequent placement of a medical instrument in the patient for the treatment;
(b) identifying the body structure in the first 3D image data;
(c) during the treatment, obtaining second 3D image data of the patient with an x-ray system that enable a spatially accurate association of the second 3D image data with the first 3D image data;
(d) spatially accurately associating the body structure and the second 3D image data with each other;
(e) during placement of the surgical instrument in the patient during the treatment, generating at least two x-ray images of the patient with the instrument in the patient using said x-ray system from respectively different directions in a known position relative to the second 3D image data; and
(f) spatially accurately associating the instrument and the body structure with each other using said known relative position and said second 3D image data, and displaying at least the surgical instrument and the body structure in a common image.

2. A method as claimed in claim 1 wherein step (a) comprises generating said first 3D image data with a stationary imaging system selected from the group consisting of computed tomography systems and magnetic resonance tomography systems.

3. A method as claimed in claim 1 wherein step (c) comprises obtaining said second 3D image data using a 3D x-ray C-arm.

4. A method as claimed in claim 3 wherein step (e) comprises acquiring said x-ray images with said 3D x-ray C-arm without displacement thereof from step (c).

5. A method as claimed in claim 1 wherein step (f) comprises mixing said body structure into at least one of said x-ray images obtained in step (e).

6. A device for displaying a surgical instrument during placement thereof in a patient during a treatment, comprising the steps of:
a control unit having an interface and a display connected thereto, said interface importing first 3D image data of a patient, obtained before a medical treatment, showing a body structure of the patient relevant for subsequent placement of a medical instrument in the patient for the treatment, said control unit automatically identifying the body structure in the first 3D image data;
an x-ray system that, during the treatment, obtains second 3D image data of the patient that enable a spatially accurate association by said control unit of the second 3D image data with the first 3D image data;
said control unit spatially accurately associating the body structure and the second 3D image data with each other;
said x-ray system, during placement of the surgical instrument in the patient during the treatment, generating at least two x-ray images of the patient with the instrument in the patient from respectively different directions in a known position relative to the second 3D image data; and
said control unit spatially accurately associating the instrument and the body structure with each other using said known relative position and said second 3D image data, and causing at least the surgical instrument and the body structure to be displayed in a common image at said display.

7. A device as claimed in claim 6 wherein said x-ray system is a 3D x-ray C-arm.
